(19) United States
(12) Patent Application Publication
(10) Pub. No.: US 2025/0134452 A1
(43) Pub. Date: May 1, 2025

(54) METHODS AND SYSTEMS FOR MONITORING SLEEP ACTIVITY AND PROVIDING A TREATMENT

(71) Applicant: Somnology, Inc., Redwood City, CA (US)

(72) Inventors: Melissa S. LIM, San Francisco, CA (US); Patrick J. YAM, Portola Valley, CA (US); Charles L. HAMILTON, Fort Bragg, CA (US); Minqiao JIN, Palo Alto, CA (US); Brian K. YAM, Portola Valley, CA (US)

(21) Appl. No.: 18/909,740

(22) Filed: Oct. 8, 2024

Related U.S. Application Data
(63) Continuation of application No. PCT/US2023/023742, filed on May 26, 2023.
(60) Provisional application No. 63/346,720, filed on May 27, 2022.

(51) Int. Cl. A61B 5/00; A61B 5/02; A61B 5/1455; G16H 40/67; G16H 50/30

(52) U.S. Cl. CPC A61B 5/4815; A61B 5/02; A61B 5/14551; A61B 5/6801; A61B 5/7267; A61B 5/7275; G16H 40/67; G16H 50/30; A61B 5/7475

(57) ABSTRACT

The present disclosure provides methods and systems for monitoring sleep activity. The method may comprise (a) collecting data associated with the subject by a wearable device attached to a body of the subject, (b) providing the data as input to a machine learning algorithm, wherein the machine learning algorithm has been trained on a plurality of training samples, wherein a training sample of the plurality of training samples comprises (i) a plurality of vital signs and a plurality of sleep activities associated with an additional subject other than the subject, wherein the plurality of vital signs comprises oxygen desaturation index, and (ii) a label that indicates whether the additional subject has cardiovascular disease (CVD); and (c) receiving from the machine learning algorithm an electronic output on a graphical user interface, wherein the electronic output comprises a sub-output indicative of a risk that the subject has the CVD.

### CROSS-REFERENCE

This application is a continuation of International Patent Application No. PCT/US2023/023742, filed May 26, 2023, which claims priority to U.S. Provisional Patent Application No. 63/346,720, filed on May 27, 2022, which applications are incorporated herein by reference in their entirety for all purposes.

### BACKGROUND

The issue of sleep deprivation has been a long-standing problem worldwide and has resulted in various consequences. An estimated 70 million people in the U.S. and 1 billion people worldwide may suffer from sleep disorders. In 2018, the Center for Disease Control classified sleep disorders as a health epidemic that is growing at a rate of 9% per year. According to a study conducted by Harvard University, on average, each person loses 11 workdays per year due to sleep-related disorders. This loss of workdays can cost each employer approximately $1,200 to $3,100 per employee annually, as reported by a National Safety Council study.

Sleep deprivation is one of the leading causes of loss of productivity. Studies have revealed that tired workers may be 70% more likely to be involved in accidents than energetic workers. There are instances where sleep deprivation or disorders may have contributed to certain disasters, such as the Hoboken Train Station Incident, the Exxon Valdez Spill, and the Chernobyl Explosion. The impact of sleep health on worker productivity is undeniable, as evidenced by the billions of dollars lost globally on an annual basis, as well as the extreme cases of sleep deprivation-caused disasters.

The most practical solution for most sleep-related problems is to seek a doctor's consultation. However, there is a significant barrier for people to get suitable assessments and treatments due to the long waiting times for a polysomnogram (sleep test), which can range from 6 to 8 months.

### SUMMARY

Recognized herein is a need for people to have convenient access to sleep activity monitoring devices, accurate diagnoses, and appropriate treatments.

The present disclosure provides a machine learning model that may be utilized to generate a personalized sleep score for a subject, based at least in part on historical data associated with the subject. The sleep score may be used to provide the subject other another party with an assessment of the subject's sleep quality, for example (e.g., level of restfulness).

The present disclosure provides methods and systems that may generate accurate sleep score from sensor data, in some cases with the aid of a machine learning module. Alternatively or additionally, the present disclosure provides predictions, potential diagnoses, and treatment recommendations automatically to a subject. This feedback may reduce the time and cost that a subject may need to spend to improve sleep quality. In some circumstances, a remedial device may be automatically prescribed and shipped to the subject. A third party (e.g., an employer) may have visibility into a user's (e.g., an employee) sleep score and allot work schedules accordingly. For example, for pilots, railway drivers/conductors, or truck drivers, it is crucial for the employers to reschedule users who are sleep deprived from duty. The automatic identification and prescription of remedial devices mitigating sleep disorders that affect society may save lives, in addition to saving a substantial amount of money due to accidents or downtime. Even further, early treatment of certain diseases, such as cardiovascular disease (CVD), may be beneficial, and the early prescription of remedial devices may save lives.

In an aspect, the present disclosure provides a method for monitoring sleep activities of a subject, comprising: (a) collecting data associated with the subject by a wearable device attached to a body of the subject, wherein the data comprises a plurality of vital signs and a plurality of sleep activities associated with the subject; (b) providing the data as input to a machine learning algorithm, wherein the machine learning algorithm has been trained on a plurality of training samples, wherein a training sample of the plurality of training samples comprises (i) a plurality of vital signs and a plurality of sleep activities associated with an additional subject other than the subject, wherein the plurality of vital signs comprises oxygen desaturation index, and (ii) a label that indicates whether the additional subject has cardiovascular disease (CVD); and (c) receiving from the machine learning algorithm an electronic output on a graphical user interface, wherein the electronic output comprises a sub-output indicative of a risk that the subject has or is at risk of developing the CVD.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

This method for monitoring sleep activities of a subject may help identify potential cardiovascular disease risks at an early stage, allowing for timely medical intervention and treatment. By collecting data on vital signs and sleep activities, and using machine learning algorithms trained on a large dataset of similar samples, the accuracy and reliability of the risk predictions can be increased. The graphical user interface provides an easy-to-understand output for the subject, allowing them to take appropriate actions to manage their health. This method may also be used by healthcare professionals to remotely monitor patients and provide targeted interventions based on individual patient needs. Overall, the present disclosure may help improve the quality of life for individuals suffering from sleep disorders and related health issues, and may lead to better health outcomes and reduced healthcare costs.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 schematically illustrates a system that is configured to automatically generate sleep score, and provides insights into diagnoses and treatments, according to many embodiments.

FIG. 2 illustrates a flow chart of a process for monitoring sleep activities of a subject, according to many embodiments.

FIG. 3 illustrates a flow chart of a process for training a system, according to many embodiments.

FIG. 4 illustrates a graph of a coefficient operation in connection with a Linear Regression Model for tuning the sleep score equation, according to many embodiments.

FIG. 5 illustrates a tree structure in connection with a Random Forest (RF) Model for tuning the sleep score equation, according to many embodiments.

FIG. 6 illustrates a graphical user interface output of a comparison of importance score between different models, according to many embodiments.

FIG. 7 shows a schematic of a computer system that is programmed or otherwise configured to implement methods provided herein.

FIG. 8 illustrates a set of graphical user interfaces for a corresponding device, according to many embodiments.

FIG. 9 illustrates a set of graphical user interfaces for a corresponding device, according to many embodiments.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the scope of the present disclosure. It should be understood that various alternatives to the embodiments of the present disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

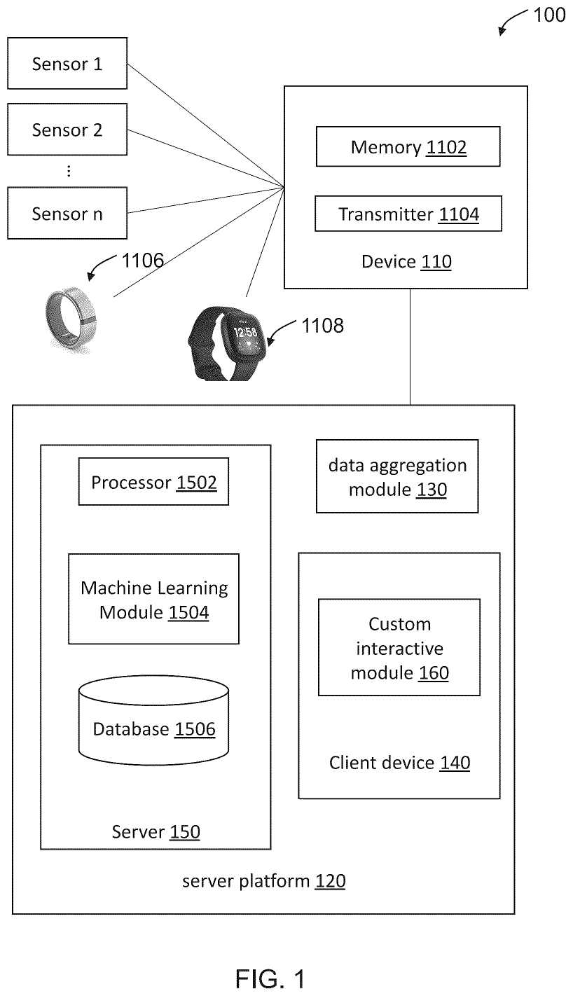

FIG. 1 schematically illustrates a system 100 that is configured to automatically provide diagnoses and treatment recommendations, according to many embodiments. The system 100 can also predict whether a subject has a risk of developing a plurality of diseases, e.g., cardiovascular disease (CVD), insomnia, restless legs syndrome, narcolepsy and sleep apnea, etc. CVD may comprise coronary artery disease (CAD), heart arrhythmias, heart failure, heart valve disease, pericardial disease, cardiomyopathy (heart muscle disease), and/or congenital heart disease, etc.

The system 100 may include a plurality of sensors, a device 110 (e.g., wearable device), a server platform 120. The system 100 and its components may be implemented by one or more computers in one or more locations.

The device 110 of the system 100 can obtain various data of a subject. The data may include a plurality of vital signs and a plurality of sleep parameters associated with a subject. The plurality of vital signs may include one or more of heart rate, respiratory rate, body temperature, peak inspiratory pressure, tidal volume, positive end-expiratory pressure, fraction of inspired oxygen, oxygen saturation, blood pressure, blood pressure, etc. The plurality of sleep parameters may include one or more of sleep duration, sleep efficiency, and sleep stage score.

The system 100 can obtain various data of a subject from a plurality of sensors. The sensors may be internet-connected sensors or connected to an internet-connected device (e.g., via a Bluetooth connection). The sensors may be pressure sensors, temperature sensors, blood oxygen sensors, blood glucose sensors, touch sensors, image sensors, ingestible sensors, motion sensors, ECG sensors, heart rate sensors, etc. The sensors may be wearable devices, such as a smart watch, a smart ring, etc. The sensors may be invasive or non-invasive sensors. The sensors may be implantable sensors.

The sensors can obtain data continuously or at predetermined increments. The predetermined increments may be based on a customized setting received from a subject or a health care provider. In some embodiments, a health care provider may be a doctor, a physician's assistance, who may review a subject's vital signs and sleep activities, and then set the increments for collecting data. In some embodiment, a health care provider may control one or more sensors to obtain data of a subject (e.g., a patient) in real-time. Alternatively or additionally, the sensors may be controlled remotely by a health care provider over a network. In some embodiments, the predetermined increments may be automatically set by a machine learning (ML) algorithm. The ML algorithm may be trained on a plurality of training samples and may be able to understand the criticality of a subject based on collected data, and then set the increments accordingly. The details of operations and functions of the ML algorithm are described elsewhere herein.

The sensors can transmit the obtained data to a device 110. The device 110 may be a computing device, with or without a display screen. Alternatively or additionally, the device 110 may be a smart device, such as a smart watch, a smart ring, etc., which comprises sensors in itself. The device 110 may include a memory 1102 and a transmitter 1104. The memory 1102 may be dynamic or static random-access memory, read-only memory, flash memory, hard drives, or the like. The memory 1102 may be configured to temporarily store the data obtained by the sensors. The transmitter 1104 may transmit the data obtained from the sensors to a server platform 120 comprising a server 150. The server platform 120 and the server 150 may be located remotely from the device 110. Alternatively or additionally, the data obtained by the sensor may be transmitted directly from the sensors to a server platform 120.

In some embodiments, the device 110 may be a smart device, such as a smart watch that may be worn around the wrist of a subject, a smart ring that may be worn around a finger of a subject. In some embodiments, the device 110 may be communicatively couple to a smart ring 1106, and/or a smart watch 1108 to transmit data. A smart watch 1108 or smart ring 1106 may monitor a subject and collect data of the subject while the subject is asleep. In some embodiments, the smart watch 1108 or smart ring 1106 may monitor the subject and collect data of the subject whenever it is being worn. The data collected by the smart watch 1108 or smart ring 1106 comprises but not limited to one or more of sleep duration, sleep efficiency, sleep stage score, heart rate, respiratory rate, body temperature, peak inspiratory pressure, tidal volume, positive end-expiratory pressure, fraction of inspired oxygen, oxygen saturation, blood pressure, etc.

In some embodiments, to achieve lower power consumption and smaller size for the device 110, the transmitter 1104 may transmit a subset of the obtained data to the server platform 120. In some embodiments, the device 110 may be configured to instruct only a subset of the sensors to collect data of the subject, and temperately disable other subsets of the sensors, based on the data predetermined to collect and/or obtain. For example, if for a particular type of sleep score calculation, only data from Sensors A, B, and C is needed, the device may instruct Sensors A, B, and C to collect data, and temperately disable the rest of the sensors. This operation may aid in saving power consumption for the device 110 and may also reduce the required size of the device 110.

In some embodiments, the instructions in terms of which groups of sensors may be activated may be received from the server platform 120. For example, the server platform 120 may determine and select, dynamically, combination of one or more sensors, and generate instructions to activate the selected combination of sensors. The server platform 120 may also generate instructions to deactivate the non-selected sensors. In some other embodiments, a subject/health care provider may manually select a combination of sensors and activate the selected sensors.

Alternatively or additionally, all sensors may be in operation and collect data from a patient or a subject. In some embodiments, only a subset of the collected data may be transmitted to the server platform 120. This selection operation and/or determination in terms of which subset of the collected/obtained data to transmit to the server platform 120 may be determined by the server platform 120. Alternatively or additionally, a subject or a health care provider may manually select a subset of the selected data to be transmitted to the server platform 120.

The system 100 may include a server platform 120. The server platform 120 may implement computer software, application software, system software, etc. The server platform 120 may include a data aggregation module 130, a client device 140, a server 150, and a custom interactive module 160. The data aggregation module 130 may a component of the server 150. The system 100, the sensors, the device 110, the software platform 120, the data aggregation module 130, the client device 140, the server 150, and the custom interactive module 160 may be implemented one or more computers in one or more locations.

The data aggregation module 130 can aggregate data collected by the sensors into datasets. The data aggregation module 130 can aggregate data collected by the sensors based on different schemes. For example, data associated with patient 1 may be aggregated as one dataset. In another example, data associated with patients with a certain type of disease (e.g., cardiovascular disease (CVD), sleep apnea, etc.), may be aggregated as one dataset. In yet another example, data associated with patients who are above a certain age (e.g., 65) may be aggregated as one dataset. In some cases, a health care provider may customize the schemes based on the health care provider's specific needs or understanding in terms of which sets or subsets of data may be aggregated. The data aggregation module 130 can be a stand-alone unit, or can be part of the client device 140, or part of the server 150. The input data for the data aggregation module 130 may come from the sensors directly, or from the device 110, or from the database 1502 of the server 150. The aggregated dataset may be presented to a health care provider through a client device 140.

The client device 140 may be desktop or laptop computers, electronic tablets, mobile devices, or the like. Although FIG. 1 shows only a single client device 140, the client device 140 may actually be an interconnected system of computing devices located in one or more locations (e.g., multiple terminals in a health care facility, health care providers' home in the setting of remote working, etc.). The client device 140 may have general-purpose processors, graphics processing units (GPU), application-specific integrated circuits (ASIC), field-programmable gate-arrays (FPGA), machine learning accelerators, or the like. The client device 140 may additionally have memory, e.g., dynamic or static random-access memory, read-only memory, flash memory, hard drives, or the like. The memory may be configured to store instructions that, upon execution, cause the client device 140 to display data associate with a subject (e.g., a patient), or predict whether a subject has a risk to develop a certain disease. Additionally or alternatively, the memory may be configured to store instructions that, upon execution, cause the client device 140 to monitor subjects in a home or clinic setting with aid of the collected/obtained data associated with a subject. In some cases, the client device 140 may display various user interfaces that provide information to a subject or a health care provider regarding the subjects sleep quality and potential related disease.

The client device 140 may include a custom interactive module 160. The custom interactive module 160 may enable a health care provider to create a custom diagnostic, a treatment recommendation, or treatment algorithm (e.g., using the collected data as input to the algorithm). The custom interactive module 160 may be located within the client device 140, the server 150, or be a stand-alone device. The custom interactive module 160 may be located remotely from the sensors, and/or the device 110. In some cases, the custom interactive module 160 may be implemented remotely from the software platform 120. A health care provider can use the custom interactive module 160 to create a custom diagnostic algorithm that processes a subset of the data obtained by the sensors. Additionally or alternatively, a health care provider can use the custom interactive module 160 to create a custom diagnostic algorithm that process all the data obtained by the sensors. A custom diagnostic algorithm may provide a diagnosis based on one or more of the vital signs or the sleep activity measurements associated with a subject (e.g., patient). The custom diagnostic algorithm may be created specific for a subject. In some embodiments, the custom diagnostic algorithm may be created for a particular clinic. A health care provider can select a group of subject data as input to the custom diagnostic algorithm. The input selection may be based on the health care provider's expertise in diagnosing one or more particular type of diseases. The input selection may also be based on a health care provider's past experience in diagnosing one or more particular types of diseases. The output of the custom diagnostic algorithm may be a probability of having a particular condition or disease (e.g., cardiovascular disease (CVD), sleep apnea, etc.) Alternatively or additionally, the output of the custom diagnostic algorithm may be a binary output regarding whether the subject does or does not have a certain disease.

The health care provider may use the custom interactive module 160 to create a custom treatment algorithm that processes a subset of the data obtained by the sensors. The custom treatment algorithm may also process a diagnosis associated with a subject determined by the custom diagnostic algorithm. Alternatively, a health care provider may input a diagnosis associated with a subject into the custom treatment algorithm. In some embodiments, the diagnosis associated with a subject is automatically input into the custom treatment algorithm without health care provider intervention. A health care provider may select a number of treatments for the custom treatment algorithm. Potential treatments may be curative, palliative, or preventative treatments. A health care provider may define, by utilizing the custom treatment algorithm, that a certain remedial device A or a medicine B may be given to a subject when the subject is diagnosed to have a disease C. A health care provider may also define, by utilizing the custom treatment algorithm, that a subject with certain vital signs and/or sleep activity measurements may be treatment with treatment D. In some embodiments, a health care provider may also define, by utilizing the custom treatment algorithm, that a certain group of recommendations of lifestyle changes may be appropriate to a subject, based on the diagnosis generated.

In some embodiments, the health care provider may utilize the custom interactive module 160 to provide one or more forms of interventions, including administrative interventions, to improve patient care. For example, one form of an administrative intervention may be a telehealth session, which could be recommended by the custom interactive module 160 based on the patient's sleep test results. For example, after a pre-defined amount of sleep tests (e.g., 4, 5, 6, 7, 8, 9, or 10), which would give the medical team enough sleep data to conduct trend analysis, the custom interactive module 160 may recommend an FDA-approved Home Sleep Testing (HSAT) device, a Polysomnogram test, or continued monitoring. In some embodiments, once the patient completes the recommended test, another administrative intervention in the form of a telehealth session may be recommended by the custom interactive module to discuss the results and determine next steps in the patient's care.

The custom treatment algorithm may be consecutive algorithms. The custom treatment algorithm may support a decision support function. For example, a physician may use the custom treatment algorithm to define a number of sets of rules. The set of rules may include logical operands such as “text/content search” or “greater, equal, less, not equal, in, not in, between” etc. The set of rules and the logical operands may also perform on logical groups and the rules can be defined with “and/or”. These logical operands may operate for health data (e.g., vital signs, sleep activity measurements) obtained by the sensors. For example, the decision support function may operate in the following manner: if “min, max, average, median” value of a vital sign or a sleep activity measurement continues for X time, then certain actions may be taken. Another more specific example can be: if a subject's sleep duration is below 6 hours for 3 nights, decision support function is activated. In another example, if a subject's oxygen desaturation index is below a certain degree for a certain period of time, the decision support function is activated.

A health care provider may define what action or actions the decision support function may take once activated based on the subject health data (e.g., vital signs, sleep activity measurements) obtained by the sensors. For example, a notification (e.g., SMS text or email) may be automatically generated and sent to a health care provider, such as the treating doctor or a nurse. In another example, the decision support function may take actions when certain vital signs or sleep activity measurements fulfill the criteria defined (e.g., preset) by the custom treatment algorithm. For example, a health care provider may define what actions the decision support function may take when a subject's certain vital signs are within predefined parameters. If a subject's blood oxygen level is below 80 ml/L for more than 30 seconds, then the decision support function is activated, and based on the custom treatment algorithm that has been created and approved by a doctor, a portable oxygen supply device is automatically prescribed and shipped to the subject (e.g., subject's home address provided on profile), and/or a push notification may be send to the subject regarding the instructions of usage of the oxygen supply device. The actions recommended and/or performed by the decision support function may include other actions, such as medication, examination, consultation, nurse care, or analysis on vital signs and sleep activity measurements. The actions recommended and/or performed by the decision support function are based on the real-time and recorded vital signs and sleep activity measurements associated with a subject. In some embodiments, when defining the custom treatment algorithm, a doctor's approval of actions is obtained. Based on the recommendations of the decision support function, a health care provider may take actions based on the recommendations. For examples, if the decision support function recommends administrating medicine A to the subject, a health care provider may adjust the frequency or dosage of the medicine A may be given to the subject. When the action is performed, the custom treatment algorithm may collect the outcomes of the action taken. It may also label the outcomes associated with certain actions and then feed it as training samples to the machine learning module 1504. The machine learning module 1504 may train itself and provide recommendations when a health care provider subsequently creates another custom treatment algorithm.

In some embodiments, the custom treatment algorithm may generate a diagnostic determination or a treatment recommendation automatically, without human involvement or intervention. This may further reduce the time and cost for a subject to undertake actions that mitigate the sleep related issues. In some embodiments, the treatment recommendations may comprise prescribing the subject a remedial device. The remedial device may comprise Continuous positive airway pressure (CPAP) device, dental devices, etc. In some embodiments, the sensors may collect attribute data associated with a subject, such as size of a portion of the body, for the purpose of customizing a remedial device. For example, the sensors may automatically collect a chest diameter for a subject, if a remedial patch around the chest is recommended and prescribed. In other embodiments, the device 110 may prompt a subject to manually enter certain attributes to customize a remedial device. This may provide benefits such as increasing the effectiveness and comfort of the remedial device, as it is tailored to the individual's unique body measurements. Additionally, a prompt may serve as a reminder for the subject to regularly use the device, increasing compliance and the chances of success in mitigating the sleep-related issue. Overall, the automation of the diagnostic and treatment recommendation process, as well as the customization of remedial devices, may provide a more efficient and effective solution for individuals seeking to improve their sleep health.

In some embodiments, when generating a personalized treatment plan for a patient with sleep apnea, there may be several remedial solutions that can be considered beyond the use of CPAP. For example, in addition to CPAP, a weight loss recommendation may be prescribed if the patient's BMI is over 28. In some other embodiments, dental devices, nasal dilators, nasal corticosteroid sprays, side sleeping, and a variety of surgeries ranging from correcting a deviated septum to UPPP, tongue reduction, jaw advancement, myofacial physical therapy, electrotherapy of the tongue muscles, and genioglossal nerve stimulation may be recommended to a patient. A lifestyle changes advise, such as reducing alcohol consumption, increasing exercise, and avoiding sleep deprivation, may be prescripted to a patient, based at least in part on the sleep trend analysis.

In some embodiments, the sensors are configured to collect a second set of data associated with the subject after the subject undertakes a remedial device. The second set of data may be transmitted to the server platform 120. The server platform 120 may run a comparison between the initial set of data and the second set of data to provide an effective measurement of the remedial device. The custom treatment algorithm of the custom interactive module 160 may further determine whether to provide a further diagnosis, an additional treatment recommendation, or to prescribe an alternative remedial device based on the comparison.

The server 150 may communicate with the data aggregation module 130, the client device 140, and the custom interactive module 160 over a network or a bus. The server 150 may include a processor 1502, a machine learning module 1504, and a database 1506. The processor 1502 may be a single core or multi core processor, or a plurality of processors for parallel processing. The database 1506 may store the data obtained by the sensors. The database 1506 may be centralized database, distributed database, NoSQL database, operational database, relational database, object-oriented database, graph database, cloud database, or the like. The data obtained by the sensors may be transmitted via the transmitter 1104 of the device 110 to the database 1506. The data aggregation module 130 may receive the data obtained by the sensors and transmit the aggregated datasets to the database 1506. Alternatively or additionally, the data obtained by the sensors may be transmitted directly from the sensors to the database 1506. The database 1506 may store the data obtained from the sensors as raw data, or as aggregated datasets. Alternatively, the database 1506 may store data associated with a plurality of subjects received from an outside source. The data associated with a plurality of subjects may be used as training dataset to train machine learning algorithms that are implemented in the machine module 1504. The data associated with a plurality of subjects may include a plurality of vital signs and a plurality of sleep activity measurements and a label that indicates whether the subject had a type of disease (e.g., cardiovascular disease (CVD), sleep apnea, etc.). In some cases, the label may also indicate the time period between a subject having a particular lab value and the time the subject develops a certain disease. In other cases, the label indicates the time period between a subject reaches a plurality of pre-determined criteria of the viral signs and sleep activity measurements and the time the subject develops a certain type of disease. Alternatively, the data obtained by the sensors may be updated with a label indicating whether a subject developed a type of disease (e.g., cardiovascular disease (CVD), sleep apnea, etc.), and then be provided as a training samples to the machine learning module 1504.

The machine learning module 1504 may be trained by the data associated a plurality of subjects. The training dataset may include a plurality of vital signs and a plurality of sleep activity measurements and a label that indicates whether the subject had a type of disease (e.g., cardiovascular disease (CVD), sleep apnea, etc.). The machine learning module 1504 may train a supervised machine learning algorithm. The supervised machine learning algorithm may comprise a neural network. Alternatively or additionally, the supervised machine learning module 1504 may implement classification machine learning algorithms. Classification machine learning algorithms may predict whether a subject will develop a certain disease based on the data associated with the subject. Alternatively or additionally, the supervised machine learning module 1504 may implement regression machine learning algorithms. The regression classification machine learning algorithms may predict the probability a subject will develop a certain disease based on the data associated with the subject. Other example of the machine learning algorithms may include Linear Regression, Random Forest, and Gradient Boosting, as described in further details elsewhere herein.

The output of the machine learning module 1504 may be indicative of whether a subject or a subject may develop a disease. The output may be verified by whether the subject actually develops the disease. Once verified, the output may be labeled as a training sample, and be feed back into the machine learning algorithm to further improve the machine learning algorithm.

The machine learning module 1504, in some embodiments, may be further configured to generate sleep score and/or sleep report for a subject. In some embodiments, the machine learning module 1504 may be utilized to fine tune a sleep score equation yeah order to achieve better results. For example, the weights associated with the parameters maybe fine-tuned but the machine learning algorithm implemented by the machine learning module 1504. Details of this operation are described elsewhere herein.

Alternatively or additionally, the machine learning module 1504 may generate a personalized sleep score for a subject. For example, the machine learning module 1504 make collect historical data associated with a particular subject and use the collected historical data to train a machine learning algorithm. A personalized sleep score may take into account the baseline differences between individual subjects and may be able to provide a more accurate sleep score. For example, for different subject, the weight associated with different parameter can be different. If a person naturally only needs 7 hours of sleep instead of a clinically recommended 8 hours, the weight assigned to the duration of sleep perimeter maybe lower than for other people. In some embodiments, if a person's heart rate is generally high, for example resting heart rate equals to 90 BPM, then the weight assigned to heart rate parameter in the sleep score equation may be lower than for other people. This personalization in calculating sleep score by a machine learning algorithm can provide a subject/patient with a sleep score that more accurately reflects their sleep pattern by differentiating different people's traits. In some embodiments, the sleep score may be a composite measure that incorporates various important sleep characteristics and may be used to generate a personalized sleep score for each user. For example, this personalized sleep score may be utilized to risk stratify users and determine the urgency of interventions and follow-up, which may be useful in a resource-constrained system. In some embodiments, certain user traits, such as large neck size or overweight, may affect how the algorithm utilize the sleep score to specifically treat these users. In some embodiments, these specific traits may not be contained in the personalize sleep score; these traits may be discussed in a telehealth session so that treatment interventions aimed at those traits can be considered. For example, the personalized sleep score can be used in conjunction with other patient information to tailor interventions and optimize treatment outcomes. Details of this operation are described elsewhere herein.

In some embodiments, one approach may comprise identifying differences among clients based on a variety of factors, including weight, age, and physical activity levels. For example, the needs of a professional athlete, college athlete, high performance gym goer, weekend warrior, and individuals who lead a more sedentary lifestyle may be considered. To track the difference between individuals, the methods and systems provided herein may establish a baseline for the client's sleep health and/or heart rate variability (HRV) metrics over a week, month, or longer, and compare it to the most recent sleep test they have taken. This approach may enable the systems provided herein to provide personalized recommendations based on users' individual metrics and improve user' sleep quality.

The system of FIG. 1 and its components can be implemented on one or more computing devices. The computing devices can be servers, desktop or laptop computers, electronic tablets, mobile devices, or the like. The computing devices can be located in one or more locations. The computing devices can have general-purpose processors, graphics processing units (GPU), application-specific integrated circuits (ASIC), field-programmable gate-arrays (FPGA), or the like. The computing devices can additionally have memory, e.g., dynamic or static random-access memory, read-only memory, flash memory, hard drives, or the like. The memory can be configured to store instructions that, upon execution, cause the computing devices to implement the functionality of the subsystems. The computing devices can additionally have network communication devices. The network communication devices can enable the computing devices to communicate with each other and with any number of user devices, over a network. The network can be a wired or wireless network. For example, the network can be a fiber optic network, Ethernet® network, a satellite network, a cellular network, a Wi-Fi® network, a Bluetooth® network, or the like. In other implementations, the computing devices can be several distributed computing devices that are accessible through the Internet. Such computing devices may be considered cloud computing devices.

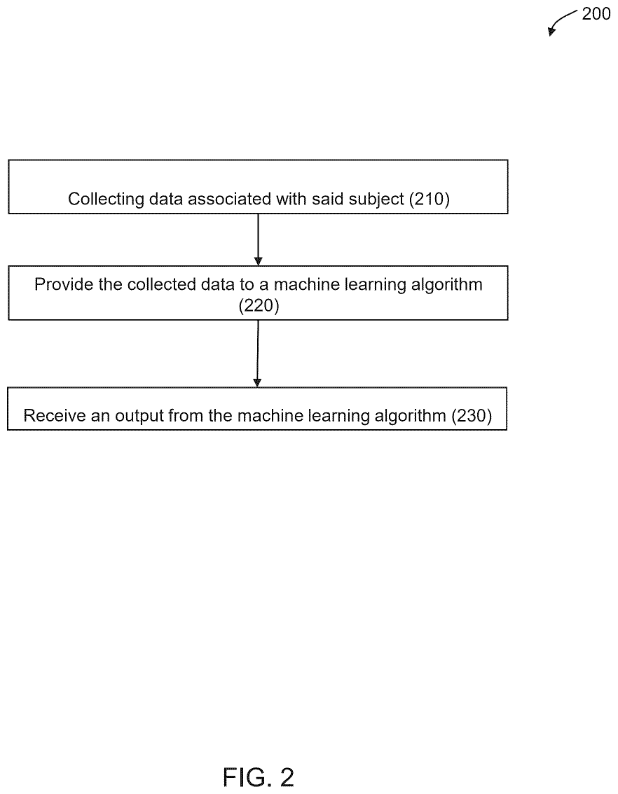

FIG. 2 is a flow chart of a process 200 for monitoring sleep activities of a subject, according to an embodiment. The process 200 can be performed by a system of one or more computers in one or more locations (e.g., the server 150 of FIG. 1).

In an operation 210, the system 100 can obtain data associated with a subject. In some embodiments, the data associated with the subject is corrected by a wearable device attached to a body of the subject. For example, the wearable device may comprise a smart watch, a smart ring, etc. The wearable device may comprise or couple with a plurality of sensors that may collect various data of the subject. The data may include a plurality of vital signs and a plurality of sleep activity measurements associated with a subject. The plurality of vital signs may comprise one or more of heart rate, oxygen intake, respiratory rate, and body temperature, and optionally peak inspiratory pressure, tidal volume, positive end-expiratory pressure, fraction of inspired oxygen, oxygen saturation, blood pressure, etc. In some embodiments, the plurality of vital signs may comprise oxygen desaturation index. The plurality of sleep activity measurements may comprise sleep duration, sleep efficiency, and sleep stage score. The data associated with a subject may be raw data or an aggregated dataset.

In an operation 220, the system 100 can provide the data associated with the subject to a trained machine learning algorithm. For example, the server 150 can cause the machine learning module 1504 to process the vital signs and/or sleep activity measurements associated with the subject. The machine learning module 1504 can then output a prediction regarding whether the subject will develop a certain disease based on vital signs and/or lab values associated with the subject. Alternatively or additionally, the machine learning module 1504 may output a sleep score of the subject based on the collected data.

In an operation 230, the server 150 can receive an output from the machine learning module 1504. The output may indicate whether the subject will develop a certain disease. In some cases, the output may indicate a probability that the subject will develop the disease. In further cases, the output may indicate a probability that the subject will develop the disease within a time period. In yet further cases, the output may include a quantitative risk score indicating the probability that the subject will develop the disease. In some other cases, the output may be an alert indicating that the subject will develop the disease. The alert is a real-time alert of a subject based at least in part on the real-time health data collected by the sensors. The server 150 can transmit the output and/or a graphical representation thereof to the client device 140 for consumption by a health care provider.

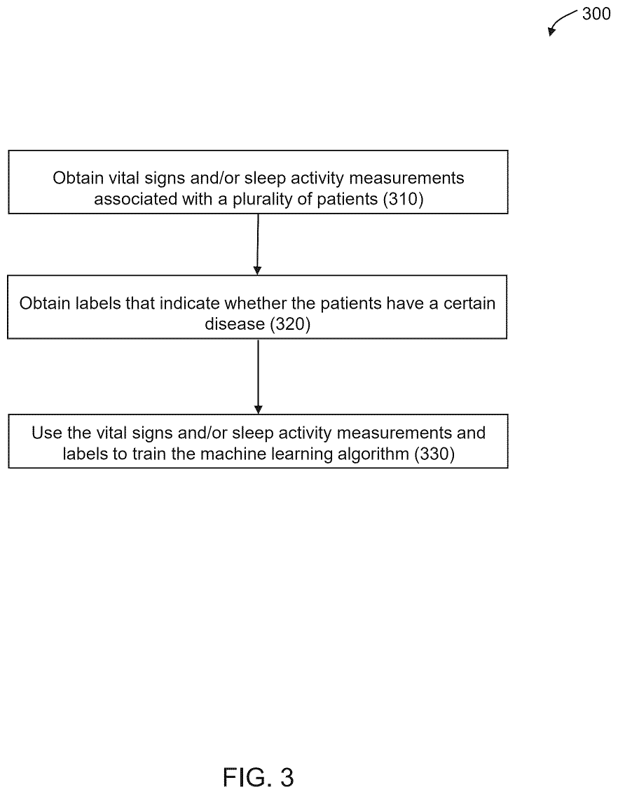

FIG. 3 is a flow chart of a process 300 for training the system 100. The process 300 can be performed by a system of one or more computers in one or more locations (e.g., the machine learning module 1504 of FIG. 1). In an operation 310, the system 100 can obtain vital signs and/or sleep activity measurements associated with a plurality of subjects. The plurality of vital signs may comprise one or more of heart rate, oxygen intake, respiratory rate, and body temperature, and optionally peak inspiratory pressure, tidal volume, positive end-expiratory pressure, fraction of inspired oxygen, oxygen saturation, blood pressure, etc. In some embodiments, the plurality of vital signs may comprise oxygen desaturation index. The plurality of sleep activity measurements may comprise sleep duration, sleep efficiency, and sleep stage score. The data associated with a subject may be raw data or aggregated dataset.

In an operation 320, the system 100 may obtain labels that indicate whether the subjects have or had a certain disease. In some cases, the label also indicates the time period between a subject reaching a certain pre-determined measurement of the vital signs and/or sleep activity and the time the subject develops a certain disease. In other cases, the label indicates the time period between a subject reaching a plurality of pre-determined criteria of the viral signs and lab values and the time the subject develops the disease. For example, a subject's oxygen desaturation index has been lower than a threshold for a certain period of time, and then this subject developed cardiovascular disease (CVD) after X number of days. The label for this training sample may reflect the time duration since the vital sign reaches the threshold to the time the subject developed the disease.

In an operation 330, the system 100 may use the vital signs and/or sleep activity measurements and labels to train the machine learning algorithm. The training may be supervised machine learning training process. In some cases, the machine learning algorithm may be trained to provide a binary prediction on whether a subject will develop a certain disease. In further cases, the machine learning algorithm may be trained to provide a probabilistic prediction that a subject will develop the disease. Alternatively or additionally, the machine learning algorithm may be trained to provide an output that indicates whether a subject will develop the disease within a time period. In these cases, the labels of the training dataset may include the time period between a subject reaching a plurality of pre-determined criteria of the viral signs and lab values and the time the subject develops the disease.

The system 100 can train the supervised machine learning algorithm by providing examples of vital signs and/or sleep activity measurements for the plurality of subjects to an untrained or partially trained version of the algorithm to generate predicted outputs that indicate whether the subject will develop a certain disease. The system can compare the predicted outputs to the known outputs (i.e., whether the subjects actually developed the disease), and if there is a difference, the system can update the parameters of the supervised machine learning algorithm. The supervised machine learning algorithm may be a regression algorithm, a support vector machine, a decision tree, a neural network, a Random Forest, and Gradient Boosting or the like. The supervised machine learning algorithm may be a binary classifier that predicts whether or not the patient will develop the disease. In some cases, the system may map the probabilistic risk score to a qualitative risk category. Alternatively, the supervised machine learning algorithm may be a multi-class classifier that predicts a qualitative risk category directly.

### Sleep Score Generation

As described elsewhere herein, the sleep score generated is not only an assessment of sleep quality but may also be an indicator of various diseases. In some embodiments, the sleep score may be used to predict cardiovascular disease (CVD). Various parameters, variables are selected to calculate a sleep score. In some embodiments, the variable may comprise: “avgsat” which denotes average saturation, “ahi_a0h3a” which denotes Apnea Hypopnea Index (AHI) at certain measurement of hour, “SlpEffP” which denotes sleep efficiency, and “TimeBedP” which denotes time in bed. In some embodiments, AHI corresponds to oxygen desaturation index (ODI) in the sleep score measurement as the measurement of the oxygen intake level.

A machine learning algorithm implemented by, for example, the machine learning module 1504 may be utilized to fine-tune the weights associated with each parameter (i.e., variables). For example, the equation may be:

$$Sleep score = sleep\_duration \_score * 0.1 + SE\_score * 0.3 + sleep\_stage \_score * 0.02 + ODI\_score * 0.15 + t90\_score * 0.15 + heart\_rate \_score * 0.28 + ( - wake\_time \_score * 0.1 )$$

In some embodiments, the data set may be obtained from various sources, for example, third party health data provider. In some embodiments, these third-party health data providers may be publicly accessible health data provider (e.g., government health data database), commercial health data provider (e.g., fitness and health data tracking companies), etc. The equation may consider the following parameters:
1. The Sleep Duration variable. In some embodiments, the Sleep Duration variable may be initially assigned with a weight of 0.1. In some embodiments, if sleep duration is between 7-9, hours, it may be given a 100% of its allotted score, giving it a 0.1 weight. In some examples, for every 30 minutes less than 7 hours and more than 9 hours, it may earn a penalty of 5% out of the score.
2. The Sleep Efficiency. In some embodiments, the Sleep Efficient is defined by total sleep time divided by total time in bed. In some embodiments, the percentage of the sleep efficiency may be assigned with a weight of 0.3.
3. The Sleep Stage Score. In some embodiments, the Sleep Stage Score may be assigned with a weight of 0.02. For example, if deep sleep/total_time_in_bed equals to 15%-20%, if REM/total time in bed is 20%-25%, and if light sleep/total_time_in_bed 50-60%, then the Sleep Stage Score may earn a 100% of its allotted score, meaning this variable is given the full 0.02 weight. In some examples, for every 5% off the percentages above, the variable may be deducted by 5%. Data collected via wearable devices may present lower degree of accuracy in monitoring sleep stage comparing to clinical-grade monitoring devices. Therefore, in some embodiments, this sleep stage score is assigned with a lower weight comparing to data collected via clinical-grade monitoring devices. This mechanism further improves the accuracy of sleep score by assigning lower weight to less reliable data.
4. Heart Rate. In some embodiments, Heart Rate (HR) is assigned with a weight of 0.28. For example, for a HR with a beats per minute (bpm) between 50-100, the full score is earned. For a HR below 50 or beyond 100 by every 10 bpm, a 5% deduction may apply.
5. Oxygen desaturation index (ODI). In some embodiments, ODI is assigned with a weight of 0.15. In some embodiments, if there is no ODI3 events (e.g., oxygen level dropped beyond 3%), then the full score is earned. For every 5 occurrences of ODI3 events, the ODI score may be deducted by 5%.
6. T90. In some embodiments, T90 is assigned a weight of 0.15. T90 measures the oxygen intake level that is under 90%. Medically, it shows great concerns if the oxygen level is below 90% for a certain amount of time. In some embodiments, for every 10% time out of total sleep time that oxygen level intake less than 90%, there may be a deduction of 5%.
7. Awake Time Penalty. In some embodiments, penalty reflects time of awake time. The Awake Time Penalty is assigned with a weight of −0.1. For example, if awake time is 0, then the value may be 0, meaning there is no penalty. In some embodiment, for every 20 minutes awake, a 5% of penalty may be imposed.

The above total weights assigned to the first 6 variables/parameter equals to 1, and the final score may be calculated by minus the penalty. The above calculation is presented merely as an example; different allocation and score calculation mechanism may be utilized to calculate this score.

### Machine Learning to Modify the Score

In some embodiments, the weights for each parameter/variable in the equation is fine-tuned using machine learning algorithm. A method is derived to calculate the overall performance of sleep quality, evaluated using public data published by third party health data and sleep data provider. Various methods in machine learning were used to fine tune and analyze the parameters of the data points.

### Linear Regression Model

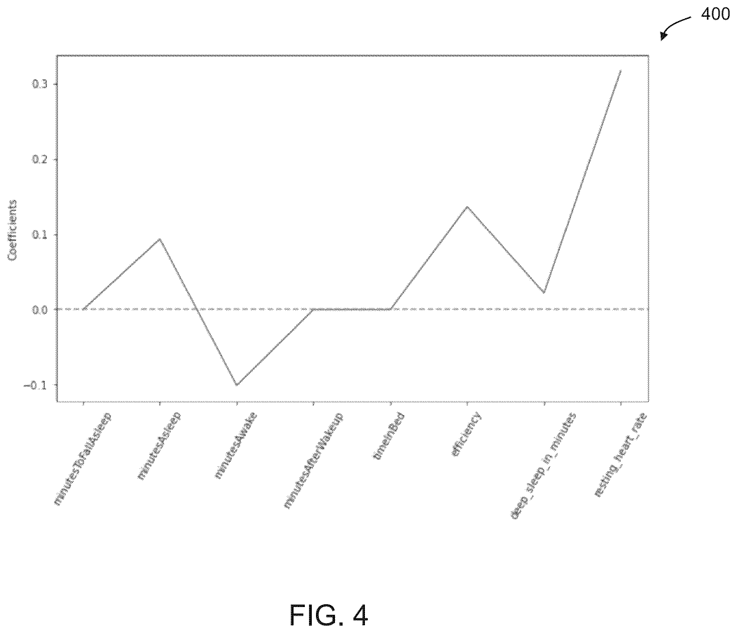

As shown in FIG. 4, a coefficient operation is utilized to investigate the parameters. In some embodiments, with the given score of sleep that third party provider calculated, a coefficient operation is utilized to investigate which parameters are the most important ones among the parameters that the third-party provider provided in their dataset. Among the parameters of “minutes to fall asleep”, “minutes asleep”, “minutes awake”, “minutes after wake up”, “minutes in Bed”, “efficiency”, “deep sleep in minutes” and “resting heart rate”, the ones that are more important may be “minutes asleep, “efficiency” and “resting heart rate” based on the coefficient operation. In which, the efficiency (sleep efficiency, SE) is calculated as total sleep time/total time in bed. In some embodiments, with the weights and features of the parameters, Linear Regression is performed to examine the data. The accuracy of using Linear Regression is 96.67% with a R\hat{ }2 square is 0.7169.

Based on the above initial analysis, the sleep score equation is formulated as following:

$$Sleep\_score = sleep\_duration \_score * 0.1 + SE\_score * 0.3 + sleep\_stage \_score * 0.02 + ODI\_score * 0.3 + heart\_rate \_score * 0.28 - wake\_time * 0.1$$

In the above equation, sleep duration corresponds to the “minutes asleep”, SE is the sleep efficiency, sleep stage corresponding to the “deep sleep in minutes” and wake time corresponding to minutes awake, and heart rate corresponding to “resting heart rate”.

### Random Forest Model

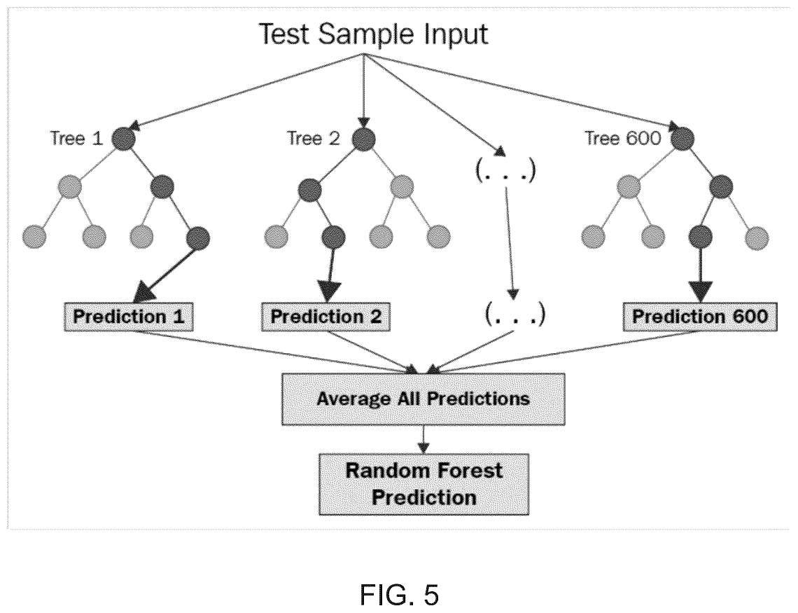

As shown in FIG. 5, a Random Forest (RF) Model may be utilized to further fine tune the weights associated with the parameters for the sleep score equation. In some embodiments, hyperparameter optimization is introduced. To avoid an overfitting that may be caused by the Linear Regression model, the RF model may randomly select each prediction from each different tree, as shown in FIG. 5. In some embodiments, all the predictions may be averaged out to get the RF prediction.

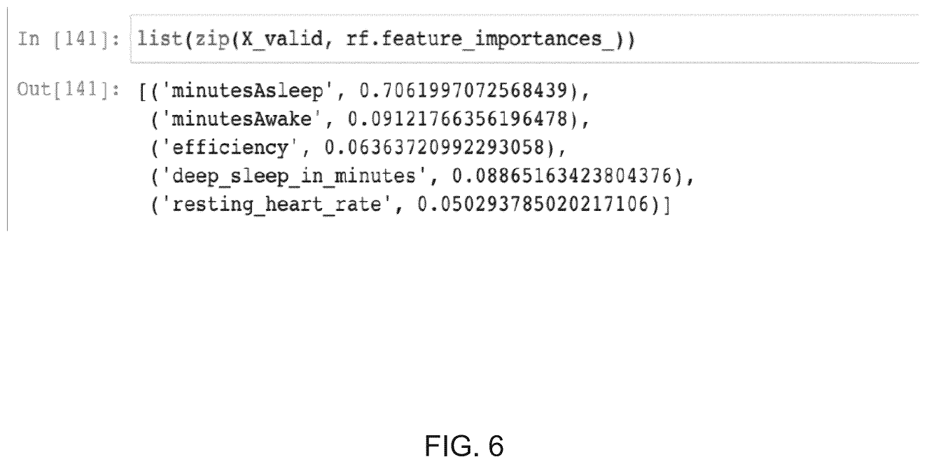

Under this RF model, as shown in FIG. 6, it shows that “minutes asleep” takes more weight compared to the Linear Regression model where “resting heart rate” is the most important feature.

In some embodiments, under the adjustment by introducing parameters and fine tuning the model, the “minutes asleep” parameter is found out to be more important than the “resting heart rate” parameter. Therefore, the sleep score equation is modified to adjust the weight distribution that reflects the newly discovered result by RF. See as the following:

$$sleep\_score = sleep\_duration \_score * 0.3 + SE\_score * 0.06 + sleep\_stage \_score * 0.06 + ODI\_score * 0.3 - wake\_time \_score * 0.1$$

### Gradient Boosting Model

In some embodiments, another model, i.e., the Gradient Boosting Model is utilized. The Gradient Boosting Model learns from its past mistake and minimizes a cost function through gradient descent. In each round of training, the weak learner (decision tree) makes a prediction, which is compared to the actual outcome. The distance between prediction and actual outcome may represent the error of the model. The errors can then be used to calculate the gradient. The Gradient Boosting Model may be built with an evaluation system established. The accuracy increases by 1% and jumps to 95.57% compared to the RF model. In some embodiments, the fit may decrease due to the other models used during the process being potentially overfitting.

### Validation and Fine Tuning of the Weights

In some embodiments, in order to validate the result, a K-fold cross validation is introduced. The operation may be split into training and validation tests multiple times, each time on different subsets of the data, train and evaluate the models each time and examine the average performance of the models across multiple evaluations. In some embodiments, another model to fine tune the result is to use Randomized Grid Search Cross-Validation. Random Search means that instead of trying out all possible combinations of hyperparameters the algorithm randomly chooses a value for each hyperparameter from the grid and evaluates the model using that random combination of hyperparameters. The way to achieve this is to define a function that compares the cross-validation performance of a set of predetermined models. Table 1 may reflect the accuracy for the above different three models and here is the comparison:

TABLE 1

| | Linear | | Extreme Gradient |
|---|---|---|---|
| | Regression | Random Forest | Boosting |
| Mean Absolute Error | 3.5534 | 3.5808 | 3.9629 |
| Mean Squared Error | 21.5618 | 22.7385 | 24.5243 |
| R\hat{ }2 | 0.6700 | 0.6520 | 0.6247 |
| Accuracy | 94.6884 | 94.6467 | 94.2521 |
| | Linear | | Extreme Gradient |
| | Regression | Random Forest | Boosting |
| Mean Absolute Error | 3.5534 | 3.5808 | 3.9629 |
| Mean Squared Error | 21.5618 | 22.7385 | 24.5243 |
| R\hat{ }2 | 0.6700 | 0.6520 | 0.6247 |
| Accuracy | 94.6884 | 94.6467 | 94.2521 |

In Table 1, the accuracy of the Linear Regression (LR) is the highest among the three of the models. This model may be the best fit for this particular dataset. Decision trees support nonlinearity, where LR supports only linear solutions. When there are a large number of features with less datasets (with low noise), LR may outperform Decision trees/random forests. In general cases, Decision trees will have better average accuracy. In this case, LR fits the best and the model used off of the third-party public database may follow a linear fit.

### Final Equation and Component Demonstration

In some embodiments, once the best fitted model is ascertained for the dataset, the weights of the model fitting by the third-party Public data can be viewed and the importance of the parameters/variables are determined based on the features after the confirmation of LR. With the research of oxygen intake level and cardinal vascular diseases, the T90 parameter (i.e., the oxygen intake level) below 90% is introduced into the equation. The data suggests that T90 and ODI are at the same important level, the weights for each parameters are evenly distributed at a 0.15 for each for the two parameters.

The final sleep score equation is presented below:

$$Sleep score = sleep\_duration \_score * 0.1 + SE\_score * 0.3 + sleep\_stage \_score * 0.02 + ODI\_score * 0.15 + t90\_score * 0.15 + heart\_rate \_score * 0.28 + ( - wake\_time \_score * 0.1 )$$

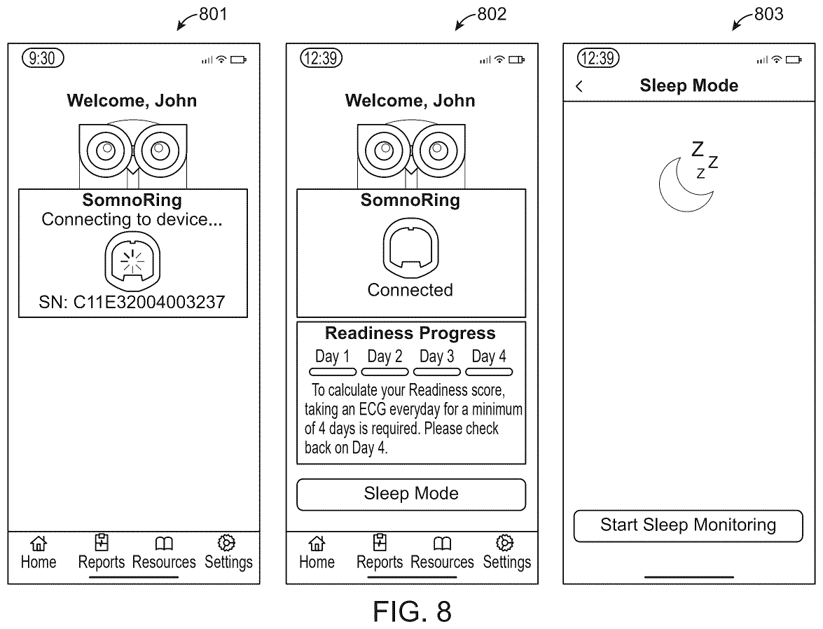

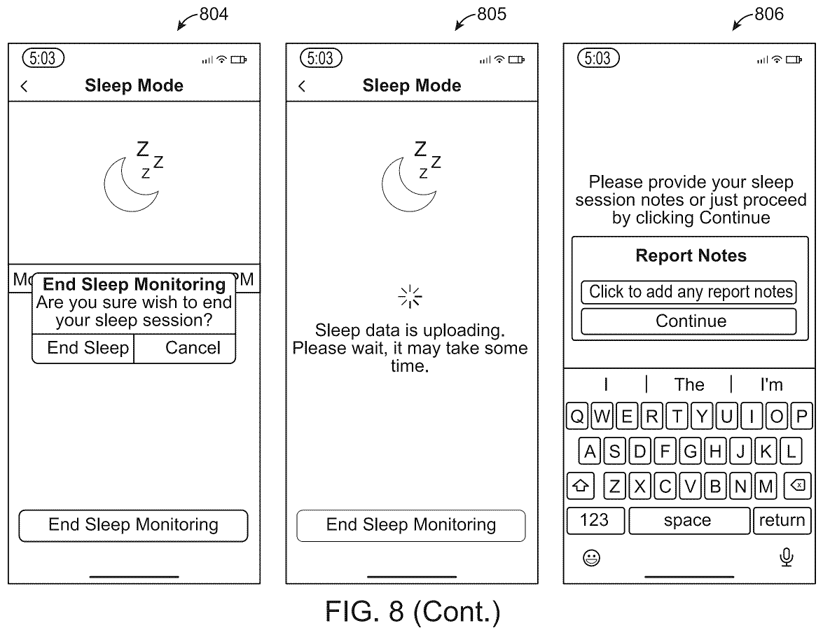

FIG. 8 illustrates a set of graphical user interfaces for a corresponding device (e.g., device 110 of FIG. 1), according to many embodiments. As shown in FIG. 8, the set of graphical user interfaces (GUIs) 801 to 806, as depicted in FIG. 8, can be utilized for various purposes such as onboarding a user, guiding them through the hardware setup process, and providing instructions for initiating sleep monitoring on the ring 1106 or a smart watch 1108 (e.g., shown in connection with FIG. 1). GUIs 801 and 802 illustrate a on boarding process, and the instruction to the user for generating a sleep score. For example, once a hardware (e.g., a ring 1106) is connected with the device 110 by the aid of GUI 801, the GUI 802 may provide instruction to the user, such as “to calculate your readiness score, taking an ECG every day for a minimum of 4 days is required. Please check back on Day 4”. This may provide clear instruction and expectation to the user, and may promote the user to perform the necessary tests to for generating the sleep report. GUIs 803-805 illustrates available operations for sleep monitoring to a user, such as start sleep monitoring, end sleep monitoring, and informative page such as informing the user that sleep data is being uploaded, according to one or more embodiments described herein. GUI 806 illustrates an input box that may allow user to generate notes regarding the sleep session.

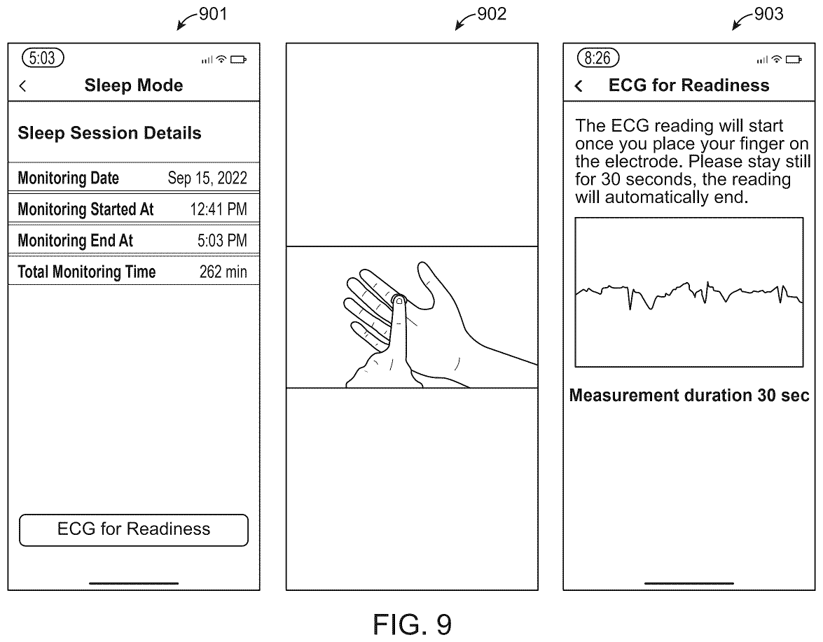

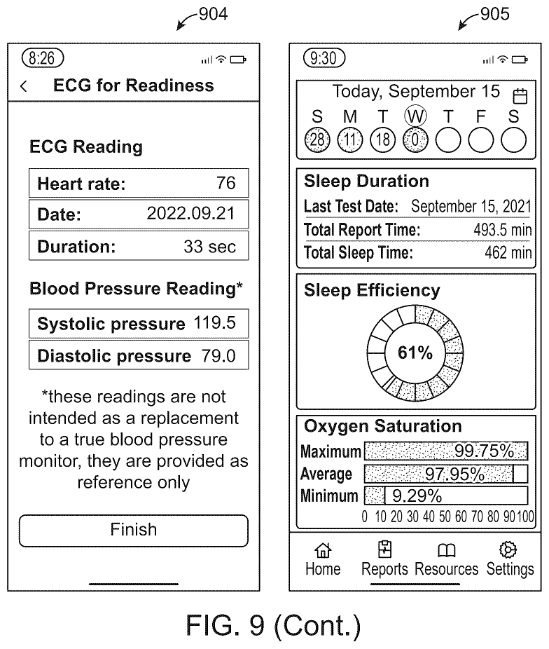

FIG. 9 illustrates a set of graphical user interfaces for a corresponding device (e.g., device 110 of FIG. 1), according to many embodiments. GUI 901 illustrates a basic sleep session details for one sleep session, and the details may comprise date, sleep started time, ended time, and duration. In some embodiments, GUI 901 may comprise a promote button to remind the user to take an ECG for readiness. In some embodiment, if the user clicks on the button, an illustration (e.g., hand position, finger position, etc.) for how to take an ECG may be provided in the next page, for example, as shown in FIG. 902. Next, as shown by GUI 903, the system may provide specific instructions on how to take an ECG. For example, the instructions may comprise the duration for testing, the location for placing one's finger, etc. In GUI 904, the ECG reading may be presented to the user. With a number of ECG data collected, (e.g., 4 nights or above), the system may be able to generate personalize trend for the user, and therefore generate comprehensive sleep report, as shown in GUI 905.

### Computer Systems

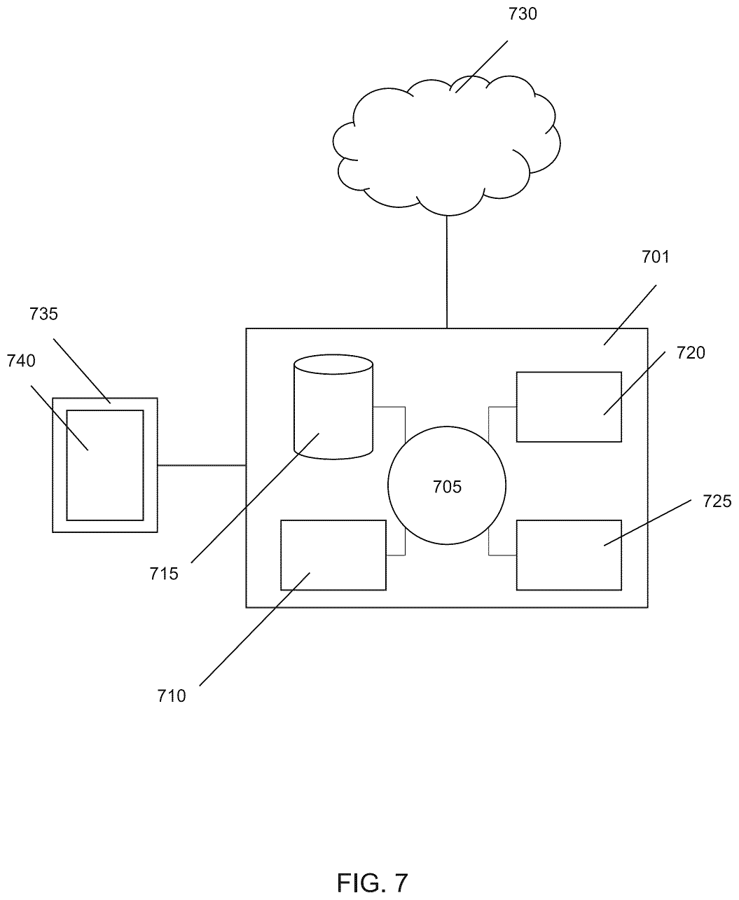

The present disclosure provides computer systems that are programmed to implement methods of the disclosure. FIG. 7 shows a computer system 701 that is programmed or otherwise configured to generate sleep score and provide diagnosis and treatment recommendations. The computer system 701 can regulate various aspects of the present disclosure. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a doctor or a doctor's assistance). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, a sleep score report. Examples of UI's include, without limitation, a graphical user interface (GUI) and web-based user interface.

What is claimed is:

1. A method for monitoring sleep of a subject, the method comprising:
(a) collecting data associated with said subject during sleep, wherein said data comprises a plurality of vital signs and a plurality of sleep parameters associated with said subject;
(b) providing said data as input to a machine learning algorithm, wherein said machine learning algorithm has been trained on a plurality of training samples, wherein a training sample of said plurality of training samples comprises (i) a plurality of vital signs and a plurality of sleep parameters associated with an additional subject other than said subject, wherein said plurality of vital signs comprises an oxygen desaturation index, and (ii) a label that indicates whether said additional subject has a cardiovascular disease (CVD); and
(c) receiving from said machine learning algorithm an electronic output on a graphical user interface, wherein said electronic output comprises a sub-output indicative of a risk that said subject has or is at risk of developing said CVD.

2. The method of claim 1, further comprising, subsequent to (c), generating a diagnostic determination or a treatment recommendation.

3. The method of claim 2, wherein the treatment recommendation comprises prescribing said subject with a remedial device.

4. The method of claim 3, further comprising, subsequent to said subject undertaking said remedial device, collecting additional data about said subject, and providing a comparison between said data and said additional data of said subject.

5. The method of claim 1, wherein said machine learning algorithm generates a sleep score for said subject based at least in part on said data.

6. The method of claim 5, wherein said machine learning algorithm uses at least said data to adjust a sleep score algorithm by adjusting a plurality of weights associated with said plurality of said vital signs and said sleep parameters.

7. The method of claim 1, wherein said machine learning algorithm uses at least a Linear Regression model, Random Forest model, or Gradient Boosting model.

8. The method of claim 1, wherein said electronic output comprises a probability that said subject will develop the CVD within a time period.

9. The method of claim 1, wherein said plurality of vital signs comprises one or more members selected from the group consisting of heart rate, oxygen intake, respiratory rate, and body temperature.

10. The method of claim 1, wherein said plurality of sleep parameters comprises one or more members selected from the group consisting of sleep duration, sleep efficiency, and sleep stage score.

11. The method of claim 1, wherein said data is collected using a wearable device attached to a body of the subject.

12. The method of claim 1, wherein the CVD comprises one or more members selected from the group consisting of coronary artery disease (CAD), heart arrhythmias, heart failure, heart valve disease, pericardial disease, cardiomyopathy (heart muscle disease), or congenital heart disease.

13. A system for monitoring sleep for a subject, the system comprising:
a plurality of sensors configured to collect data of said subject;
a platform configured to receive and process said data, wherein said platform is configured to (i) enable a health care provider to create a treatment algorithm that processes at least a subset of said data from said plurality of sensors to generate a treatment recommendation, and (ii) transmit said treatment recommendations to an electronic device of said subject or another subject; and
wherein said plurality of sensors is configured to collect an additional set of data of said subject after said subject undertakes said treatment recommendation, and said platform is further configured to provide a comparation of said data and said additional set of data of said subject.

14. The system of claim 13, further comprising a wearable device housing at least some of the plurality of sensors and configured to be one or more of worn or attached to a body of the subject during sleep of the subject.

15. The system of claim 13, wherein the treatment algorithm comprises a machine learning algorithm.

16. The system of claim 15, wherein said machine learning algorithm uses at least a Linear Regression model, Random Forest model, or Gradient Boosting model.

17. The system of claim 13, wherein the treatment recommendation comprises prescribing said subject with a remedial device.

18. The system of claim 13, wherein the collected data comprises one or more members selected from the group consisting of heart rate, oxygen intake, respiratory rate, and body temperature sleep duration, sleep efficiency, and sleep stage score.

19. A method for generating a sleep score for a subject, comprising:
(a) collecting a first set of data associated with said subject, wherein said first set of data comprises a plurality of vital signs and a plurality of sleep parameters associated with said subject;
(b) providing said first set of data to a sleep score algorithm to generate a first sleep score;
(c) providing said first set of data and said first sleep score to a machine learning algorithm, wherein said machine learning algorithm adjusts said sleep score algorithm at least in part by analyzing said first set of data and said first sleep score over time, to yield an adjusted sleep score algorithm;
(d) collecting a second set of data associated with said subject; and
(e) providing the second set of data to said adjusted sleep score algorithm to generate a second sleep score.

20. The method of claim 19, wherein in (c), said machine learning algorithm analyzes said first set of data and said first sleep score over time to create a plurality of traits associated with said subject, and adjust said sleep score algorithm for said subject based at least in part on said plurality of traits, to yield said adjusted sleep score algorithm.

21. The method of claim 19, wherein in (e), said second sleep score may be displayed to said subject with a notation indicative of a personalization nature of said second sleep score.

22. The method of claim 19, wherein the plurality of vital signs comprises one or more members selected from the group consisting of heart rate, oxygen intake, respiratory rate, and body temperature.

23. The method of claim 19, wherein said plurality of sleep parameters comprises one or more members selected from the group consisting of sleep duration, sleep efficiency, and sleep stage score.

24. A method for identifying a health or physiological state of a subject, said method comprising:
(a) using a mobile electronic device attached to a body of said subject to collect data during a period in which said subject is asleep;
(b) using a machine learning algorithm to process said data to identify said health or physiological state of said subject; and
(c) outputting a report indicative of said health or physiological state of said subject on a user interface of an electronic device of said subject or another subject.

25. The method of claim 24, wherein said mobile electronic device is attached to a wrist or a finger of said body of said subject.
